(19) United States
(12) Patent Application Publication
(10) Pub. No.: US 2011/0306884 A1
(43) Pub. Date: Dec. 15, 2011

(54) ULTRASONIC DIAGNOSTIC APPARATUS

(76) Inventors: Shunichiro Tanigawa, Tokyo (JP); Mayumi Ito, Tokyo (JP)

(21) Appl. No.: 13/157,222

(22) Filed: Jun. 9, 2011

(30) Foreign Application Priority Data
Jun. 11, 2010 (JP) 2010-133849

(51) Int. Cl. A61B 8/13

(52) U.S. Cl. 600/443

(57) ABSTRACT

An ultrasonic diagnostic apparatus includes a physical quantity calculation unit configured to calculate a physical quantity relating to an elasticity of body tissue based on ultrasound echo data, a physical quantity average unit configured to calculate an average value of physical quantities in an elastic image of the body tissue generated based on the calculated physical quantity, a comparison value calculation unit configured to calculate a comparison value for each pixel by comparing a physical quantity for each pixel of the elastic image with the average value, and an index value calculation unit configured to calculate an index value relating to an elasticity of a predetermined region in the elastic image based on the comparison values.

### CROSS REFERENCE TO RELATED APPLICATIONS

This application claims the benefit of Japanese Patent Application No. 2010-133849 filed Jun. 11, 2010, which is hereby incorporated by reference in its entirety.

### BACKGROUND OF THE INVENTION

The embodiments described herein relate to an ultrasonic diagnostic apparatus, and in particular, relates to an ultrasonic diagnostic apparatus that displays an elastic image indicating the hardness or softness of body tissue.

An ultrasonic diagnostic apparatus which combines and displays a normal B-mode image and an elastic image indicating the hardness or softness of body tissue is disclosed, for example, in Japanese Patent No. 3932482. In this type of ultrasonic diagnostic apparatus, the elastic image is generated in the following manner. First, while the body tissue is deformed e.g. by alternating compression and relaxation through an ultrasonic probe, ultrasound is transmitted and received to and from the body tissue, and an echo is acquired. Then, a physical quantity relating to the elasticity of the body tissue is calculated based on the acquired echo data. The physical quantity is converted into hue information, so that a color elastic image is generated. As the physical quantity relating to the elasticity of the body tissue, e.g. a strain of the body tissue is calculated.

### BRIEF DESCRIPTION OF THE INVENTION

Diagnosis using the elastic image is carried out according to a hue by which an attention site is displayed or a mix of multiple hues. Accordingly, diagnosis is carried out by diagnostician's subjective judgment; therefore, diagnostics results might vary among diagnosticians. For this reason, there is a need for an ultrasonic diagnostic apparatus that enables an objective and easy diagnosis.

According to a first aspect, an ultrasonic diagnostic apparatus includes a physical quantity calculation unit for calculating a physical quantity relating to elasticity of body tissue, based on echo data acquired by transmitting and receiving ultrasound to and from the body tissue, a physical quantity average unit for calculating an average value of physical quantities in an elastic image of the body tissue generated based on the physical quantity, a comparison value calculation unit for calculating a comparison value for each pixel by performing computing for comparing a physical quantity for each pixel of the elastic image with the average value, and an index value calculation unit for calculating an index value relating to elasticity of a predetermined region in the elastic image, based on the comparison value.

According to a second aspect, in the first aspect, the index value calculation unit calculates a score for each pixel by converting the comparison value using a predetermined function, and calculates an average value of scores in the predetermined region, as the index value.

According to a third aspect, in the first aspect, the index value calculation unit calculates an average value of comparison values in the predetermined region, as the index value.

According to a fourth aspect, in the first aspect, the index value calculation unit calculates an average value of comparison values in the predetermined region, and further calculates a score as the index value by converting the average value using a predetermined function.

According to a fifth aspect, an ultrasonic diagnostic apparatus includes a physical quantity calculation unit for calculating a physical quantity relating to elasticity of body tissue, based on echo data acquired by transmitting and receiving ultrasound to and from the body tissue, a gradation calculation unit for calculating a gradation for each pixel by performing gradation processing based on a physical quantity for each pixel of an elastic image of the body tissue generated based on the physical quantity, and an index value calculation unit for calculating an index value relating to elasticity of a predetermined region, based on the gradation.

According to a sixth aspect, in the fifth aspect, the index value calculation unit calculates a score for each pixel by converting the gradation using a predetermined function, and calculates an average value of scores in the predetermined region, as the index value.

According to a seventh aspect, in the fifth aspect, the index value calculation unit calculates an average value of gradations in the predetermined region, as the index value.

According to an eighth aspect, in the fifth aspect, the index value calculation unit calculates an average value of gradations in the predetermined region, and further calculates a score as the index value by converting the average value using a predetermined function.

According to a ninth aspect, an ultrasonic diagnostic apparatus includes a physical quantity calculation unit for calculating a physical quantity relating to elasticity of body tissue, based on echo data acquired by transmitting and receiving ultrasound to and from the body tissue, a first physical quantity average unit for calculating a first physical quantity average value which is an average value of physical quantities in an elastic image of the body tissue generated based on the physical quantity, a second physical quantity average unit for calculating a second physical quantity average value which is an average value of physical quantities in a predetermined region set in the elastic image, and an index value calculation unit for calculating a comparison value as an index value relating to elasticity of the predetermined region by performing computing for comparing the first physical quantity average value and the second physical quantity average value.

According to a tenth aspect, an ultrasonic diagnostic apparatus includes a physical quantity calculation unit for calculating a physical quantity relating to elasticity of body tissue, based on echo data acquired by transmitting and receiving ultrasound to and from the body tissue, a second physical quantity average unit for calculating a second physical quantity average value which is an average value of physical quantities in a predetermined region set in an elastic image of the body tissue generated based on the physical quantity, a third physical quantity average unit for calculating a third physical quantity average value which is an average value of physical quantities in a predetermined range in a distribution of physical quantities in the elastic image, and an index value calculation unit for calculating a comparison value as an index value relating to elasticity of the predetermined region by performing computing for comparing the second physical quantity average value and the third physical quantity average value.

According to an eleventh aspect, in the tenth aspect, the ultrasonic diagnostic apparatus includes a first physical quantity average unit for calculating a first physical quantity average value which is an average value of the physical quantities in the elastic image of the body tissue generated based on the physical quantity, and the third physical quantity average value is the average value of the physical quantities in the predetermined range which is set with the first physical quantity average value being as a standard.

According to a twelfth aspect, in the ninth aspect, the index value calculation unit calculates a score as the index value by converting the comparison value using a predetermined function.

According to a thirteenth aspect, in the first aspect, the ultrasonic diagnostic apparatus includes an index value comparison computing unit for performing computing for comparing index values for a plurality of predetermined regions.

According to a fourteenth aspect, an ultrasonic diagnostic apparatus includes a physical quantity calculation unit for calculating a physical quantity relating to elasticity of body tissue, based on echo data acquired by transmitting and receiving ultrasound to and from the body tissue, a first physical quantity average unit for calculating a first physical quantity average value which is an average value of physical quantities in an elastic image of the body tissue generated based on the physical quantity, a third physical quantity average unit for calculating a third physical quantity average value which is an average value of physical quantities in a predetermined range in a distribution of the physical quantities in the elastic image, and an index value calculation unit for calculating a comparison value as an index value relating to elasticity by performing computing for comparing the first physical quantity average value and the third physical quantity average value.

According to a fifteenth aspect, in the fourteenth aspect, the third physical quantity average value is the average value of the physical quantities in the predetermined range which is set with the first physical quantity average value being as a standard.

According to a sixteenth aspect, an ultrasonic diagnostic apparatus includes a physical quantity calculation unit for calculating a physical quantity relating to elasticity of body tissue, based on echo data acquired by transmitting and receiving ultrasound to and from the body tissue, a third physical quantity average unit for calculating a third physical quantity average value which is an average value of physical quantities in a predetermined range in a distribution of physical quantities in an elastic image generated based on the physical quantity, a fourth physical quantity average unit for calculating a fourth physical quantity average value which is an average value of physical quantities in a range different from a range for calculating the third physical quantity average value in the distribution of the physical quantities in the elastic image, and an index value calculation unit for calculating a comparison value as an index value relating to elasticity by performing computing for comparing the third physical quantity average value and the fourth physical quantity average value.

According to a seventeenth aspect, in the fourteenth aspect, the index value calculation unit calculates a score as the index value by converting the comparison value using a predetermined function.

According to an eighteenth aspect, in the first aspect, the ultrasonic diagnostic apparatus includes an index value display control unit for displaying the index value.

According to an aspect, an average value of physical quantities in the elastic image is calculated, and a comparison value is calculated by performing computing for comparing the average value with a physical quantity for each pixel of the elastic image. Then, an index value relating to elasticity of a predetermined region in the elastic image is calculated based on the comparison value. Therefore, it is possible to quantify the hardness or softness of the body tissue and therefore to make an objective and easy diagnosis.

Further, according to another aspect, a gradation is calculated by performing gradation processing based on a physical quantity in the elastic image, and an index value relating to elasticity of a predetermined region in the elastic image is calculated based on the gradation. Therefore, it is possible to quantify the hardness or softness of the body tissue and therefore to make an objective and easy diagnosis.

Further, according to another aspect, a comparison value is calculated as an index value relating to elasticity by performing computing for comparing the first physical quantity average value and the second physical quantity average value. Therefore, it is possible to quantify the hardness or softness of the body tissue and therefore to make an objective and easy diagnosis.

Further, according to another aspect, a comparison value is calculated as an index value relating to elasticity by performing computing for comparing the second physical quantity average value and the third physical quantity average value. Therefore, it is possible to quantify the hardness or softness of the body tissue and therefore to make an objective and easy diagnosis.

Further, according to another aspect, a comparison value is calculated as an index value relating to elasticity by performing computing for comparing the first physical quantity average value and the third physical quantity average value. Therefore, it is possible to quantify the hardness or softness of the body tissue and therefore to make an objective and easy diagnosis.

Further, according to another aspect, a comparison value is calculated as an index value relating to elasticity by performing computing for comparing the third physical quantity average value and the fourth physical quantity average value. Therefore, it is possible to quantify the hardness or softness of the body tissue and therefore to make an objective and easy diagnosis.

### BRIEF DESCRIPTION OF THE DRAWINGS

FIG. 1 is a block diagram showing one example of the schematic configuration of an ultrasonic diagnostic apparatus.

FIG. 2 is a block diagram showing the configuration of a display control unit in the ultrasonic diagnostic apparatus shown in FIG. 1.

FIG. 3 is an illustration showing a display unit on which an ultrasonic image composed of a B-mode image and an elastic image is displayed.

FIG. 4 is an illustration showing the display unit on which a predetermined region is set in the elastic image in the ultrasonic image.

FIG. 5 is a graph for explaining calculation of scores based on ratios.

FIG. 6 is an illustration showing the display unit according to a third modification of a first embodiment.

FIG. 7 is a block diagram showing the configuration of the display control unit according to the third modification of the first embodiment.

FIG. 8 is a graph for explaining calculation of scores based on gradations according to a second embodiment.

FIG. 9 is a block diagram showing the configuration of the display control unit in the ultrasonic diagnostic apparatus according to a third embodiment.

FIG. 10 is a graph for explaining calculation of scores based on ratios according to the third embodiment.

FIG. 11 is a block diagram showing the configuration of the display control unit in the ultrasonic diagnostic apparatus according to a fourth embodiment.

FIG. 12 is a diagram showing the distribution of strains in an elastic image display region.

FIG. 13 is a graph for explaining calculation of scores based on ratios according to a first modification of the fourth embodiment.

FIG. 14 is a diagram showing the distribution of strains in the elastic image display region.

FIG. 15 is a block diagram showing the configuration of the display control unit in the ultrasonic diagnostic apparatus according to a fifth embodiment.

FIG. 16 is a block diagram showing the configuration of the display control unit in the ultrasonic diagnostic apparatus according to a sixth embodiment.

FIG. 17 is a diagram showing the distribution of strains in the elastic image display region.

FIG. 18 is an illustration showing the display unit on which an image G1 composed of the B-mode image and the elastic image and an image G2 composed of only the B-mode image are displayed side-by-side.

### DETAILED DESCRIPTION OF THE INVENTION

Hereinafter, embodiments of the present invention will be described in detail with reference to the accompanying drawings.

### First Embodiment

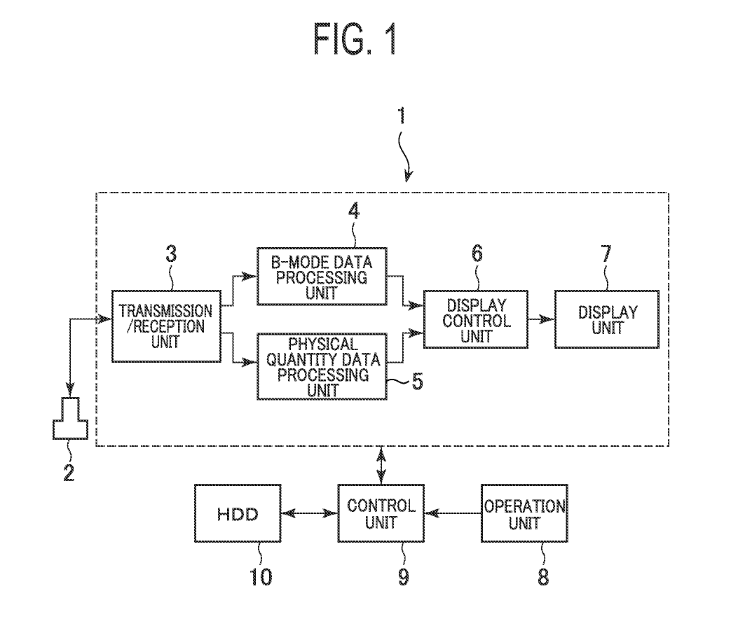

First, a first embodiment will be described with reference to FIGS. 1 to 5. An ultrasonic diagnostic apparatus 1 shown in FIG. 1 includes an ultrasonic probe 2, a transmission/reception unit 3, a B-mode data processing unit 4, a physical quantity data processing unit 5, a display control unit 6, a display unit 7, an operation unit 8, a control unit 9, and an HDD (Hard Disk Drive) 10.

The ultrasonic probe 2 transmits ultrasound to body tissue and receives its echo. An elastic image is generated as described later based on echo data acquired by transmitting and receiving ultrasound while deforming the body tissue by alternating compression and relaxation with the ultrasonic probe 2 in contact with the surface of the body tissue or applying acoustic radiation pressure from the ultrasonic probe 2 to the body tissue.

The transmission/reception unit 3 drives the ultrasonic probe 2 under a predetermined scan condition based on a control signal from the control unit 9, thereby performing ultrasonic scanning every sound ray. Further, the transmission/reception unit 3 performs signal processing such as phasing addition on the echo received by the ultrasonic probe 2. The echo data subjected to the signal processing by the transmission/reception unit 3 is outputted to the B-mode data processing unit 4 and the physical quantity data processing unit 5.

The B-mode data processing unit 4 performs B-mode processing such as logarithmic compression and envelope detection on the echo data outputted from the transmission/reception unit 3 to generate B-mode data. The B-mode data is outputted from the B-mode data processing unit 4 to the display control unit 6.

The physical quantity data processing unit 5 generates data of a physical quantity (physical quantity data) relating to the elasticity of each part of the body tissue, based on the echo data outputted from the transmission/reception unit 3. As described, for example, in Japanese Unexamined Patent Publication No. 2008-126079, the physical quantity data processing unit 5 sets correlation windows to temporally different echo data items on the same sound ray on a scanning surface, and performs correlation computing between the correlation windows, thus calculating the physical quantity relating to the elasticity and generating the physical quantity data. In this example, the physical quantity data processing unit 5 calculates a strain St as the physical quantity relating to the elasticity. The physical quantity data processing unit 5 is one example of an embodiment of a physical quantity calculation unit.

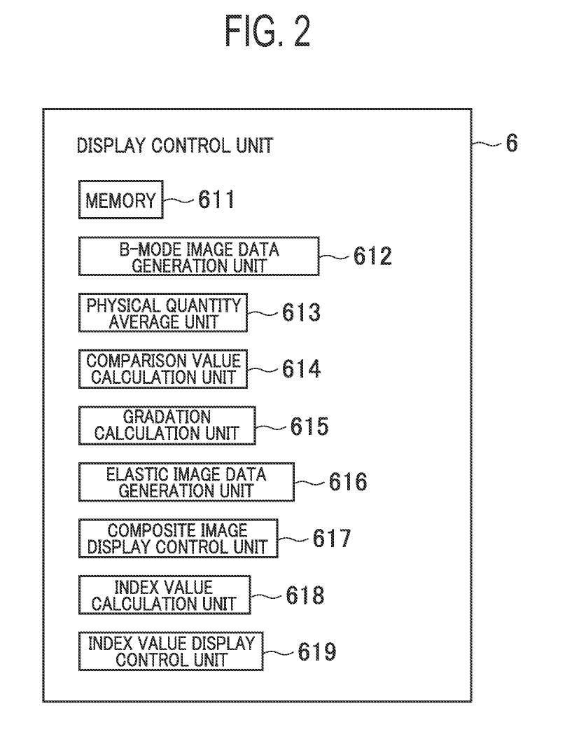

The B-mode data from the B-mode data processing unit 4 and the physical quantity data from the physical quantity data processing unit 5 are inputted to the display control unit 6. The display control unit 6 has a memory 611, a B-mode image data generation unit 612, a physical quantity average unit 613, a comparison value calculation unit 614, a gradation calculation unit 615, an elastic image data generation unit 616, a composite image display control unit 617, an index value calculation unit 618, and an index value display control unit 619, as shown in FIG. 2.

The B-mode data and the physical quantity data are stored in the memory 611. The B-mode data and the physical quantity data are stored in the memory 611 as data for each sound ray.

The memory 611 is configured with a semiconductor memory such as RAM (Random Access Memory) and ROM (Read Only Memory). The B-mode data and the physical quantity data may be stored in the HDD 10.

The B-mode image data generation unit 612 performs scan conversion of the B-mode data by a scan converter, thus converting the B-mode data into B-mode image data having brightness information according to the signal strength of the echo. The B-mode image data has, for example, 256 gradations of brightness information.

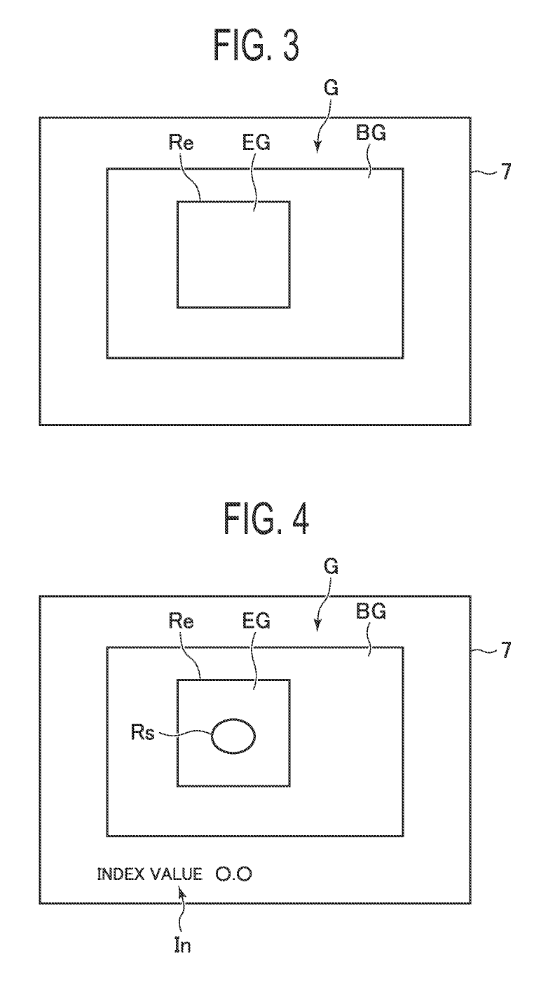

The physical quantity average unit 613 calculates an average value St_{AV} of strains in an elastic image EG (see FIG. 3) described later. Specifically, the physical quantity average unit 613 calculates the average value St_{AV} of strains St calculated for pixels in an elastic image display region Re which is a region for displaying the elastic image EG. The physical quantity average unit 613 is one example of an embodiment of a physical quantity average unit.

The comparison value calculation unit 614 performs computing for comparing the strain St for each pixel with the strain average value St_{AV}. Specifically, the comparison value calculation unit 614 calculates the ratio (Rat=St/St_{AV}) of the strain St for each pixel to the strain average value St_{AV}. The ratio Rat is calculated for each pixel. The comparison value calculation unit 614 is one example of an embodiment of a comparison value calculation unit, and the ratio Rat is one example of an embodiment of a comparison value.

The gradation calculation unit 615 performs processing for converting the ratio Rat into a value represented by N gradations (e.g., N=256) to generate gradation data composed of data of a gradation Gr for each pixel. The gradation calculation unit 615 performs gradation processing in which if the ratio Rat=1, that is, the strain St is equal to the average value St_{AV}, the gradation Gr=N/2. For example, in the case where N=256; if the ratio Rat=1, the gradation is 128. The gradation calculation unit 615 is one example of an embodiment of a gradation calculation unit.

Lower gradations Gr represent harder body tissues, and higher gradations Gr represent softer body tissues.

The elastic image data generation unit 616 performs scan conversion of the gradation data by a scan converter, thus converting the gradation data into color elastic image data having hue information according to the strain. The color elastic image data has, for example, 256 gradations of hue information.

The composite image display control unit 617 adds and combines the B-mode image data and the color elastic image data together to generate image data of an ultrasonic image G to be displayed on the display unit 7. The image data is displayed on the display unit 7 as the ultrasonic image G obtained by combining a monochrome B-mode image BG and a color elastic image EG as shown in FIG. 3. The elastic image EG is displayed in an elastic image display region Re set in the B-mode image BG in a translucent form (in a state of being transparent to the background B-mode image).

The index value calculation unit 618 calculates an index value In relating to the elasticity of a predetermined region Rs (see FIG. 4) in the elastic image EG as described later. A specific calculation method will be described later. Further, the index value display control unit 619 displays the index value In on the display unit 7. The index value calculation unit 618 is one example of an embodiment of an index value calculation unit, and the index value In is one example of an embodiment of an index value. Further, the index value display control unit 619 is one example of an embodiment of an index value display control unit.

The display unit 7 is configured with, for example, an LCD (Liquid Crystal Display), a CRT (Cathode Ray Tube), or the like. The operation unit 8 includes a keyboard and a pointing device (not shown) through which an operator inputs an instruction and information.

The control unit 9 has a CPU (Central Processing Unit), and reads a control program stored in the HDD 10 to execute functions in the units of the ultrasonic diagnostic apparatus 1.

Now, the operation of the ultrasonic diagnostic apparatus 1 of this example will be described. First, the transmission/reception unit 3 causes the ultrasonic probe 2 to transmit ultrasound to body tissue under test, and acquires its echo signal. At this time, ultrasound is transmitted and received while the body tissue is deformed. Methods for deforming the body tissue include a method for alternating compression on the tissue under test and its relaxation through the ultrasonic probe 2 and a method for applying acoustic radiation pressure to the tissue under test through the ultrasonic probe 2.

When the echo signal is acquired, the B-mode data processing unit 4 generates the B-mode data, and the physical quantity data processing unit 5 generates the physical quantity data. Further, the B-mode image data generation unit 612 generates the B-mode image data, and the elastic image data generation unit 616 generates the color elastic image data. Then, the composite image display control unit 617 displays the ultrasonic image G obtained by combining the B-mode image BG based on the B-mode image data and the elastic image EG based on the color elastic image data on the display unit 7.

Now, the generation of the color elastic image data will be described in detail. In the generation of the color elastic image data, the physical quantity average unit 613 calculates the average value St_{AV} of the strains in the elastic image display region Re, based on the physical quantity data composed of data of the strain for each pixel. Then, the comparison value calculation unit 614 calculates the ratio (Rat=St/St_{AV}) for each pixel. Then, the gradation calculation unit 615 generates the gradation data based on the ratio Rat, and the elastic image data generation unit 616 generates the color elastic image data based on the gradation data.

When the predetermined region Rs is set in the elastic image EG in the elastic image display region Re with the ultrasonic image G displayed as shown in FIG. 4, the index value In relating to the elasticity of the predetermined region Rs is displayed on the display unit 7. The predetermined region Rs is set so as to include a region presumed to be a tumor for example. The operator sets the predetermined region Rs, using the pointing device or the like of the operation unit 8.

The calculation and display of the index value In will be described in detail. The index value calculation unit 618 converts the ratio Rat for each pixel into a score SCp using a predetermined function F1, thus calculating the score SCp for each pixel. The score SCp for each pixel may be stored in the memory 611 or the HDD 10.

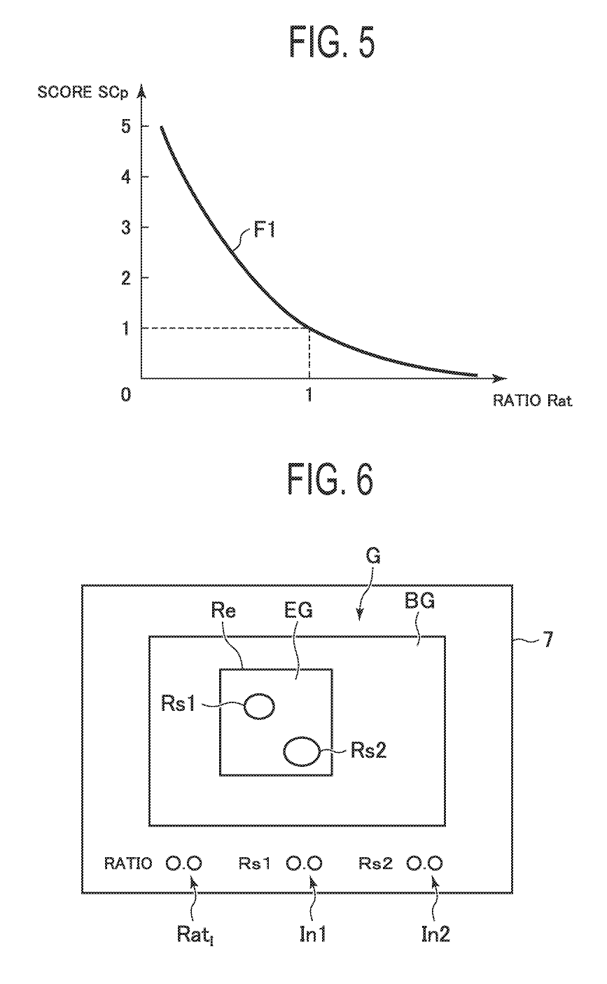

The function F1 is a function in which the ratio Rat is converted into the score SCp in a predetermined numerical range. In this example, the ratio Rat is converted into the score SCp in the range of 0.1 to 5, using the function F1 as shown in FIG. 5. In this example, lower scores SCp represent softer body tissues, and higher scores represent harder body tissues. However, the numerical range of scores SCp is one example, and is not limited to the range of 0.1 to 5 (the same applies to the following embodiments).

More specifically, the function F1 is a function in which if the ratio Rat is 1, that is, the strain St is equal to the strain average value St_{AV}, the score SCp is 1. Further, the function F1 is a function in which the ratio Rat of lower than 1 is converted into the score SCp of higher than 1 and not higher than 5 and the ratio Rat of not lower than 1 is converted into the score SCp of not lower than 0.1 and not higher than 1. Therefore, the function F1 is a function in which scores SCp are distributed more minutely in the range of ratios Rat lower than 1 than in the range of ratios Rat not lower than 1.

The range of ratios Rat lower than 1 is the range of strain values St lower than the average value St_{AV}, and is the range harder than average. A tumor is harder than a normal tissue, and there is a difference in hardness between a benign tumor and a malignant tumor. Accordingly, by the function F1 in which scores are distributed more minutely in the range of ratios Rat lower than 1 than in the range of ratios Rat not lower than 1 as described above, it becomes possible to observe the tumor more minutely.

The index value calculation unit 618 calculates the average value SC_{AV} of scores in the predetermined region Rs, based on the score SCp for each pixel. The average value SC_{AV} is the index value In. Then, the index value display control unit 619 displays the average value SC_{AV} as the index value In on the display unit 7.

According to the ultrasonic diagnostic apparatus 1 of this example, since the average value SC_{AV} is displayed as the index value In relating to the elasticity, it is possible to quantify and indicate the hardness or softness of the body tissue. Therefore, it is possible to make an objective and easy diagnosis.

Next, modifications of the first embodiment will be described. First, a first modification will be described. In the first modification, the index value calculation unit 618 calculates the average value Rat_{AV} of ratios Rat calculated for pixels in the predetermined region Rs, as the index value In. Then, the index value display control unit 619 displays the average value Rat_{AV} as the index value In relating to the elasticity (hardness or softness) of the body tissue on the display unit 7.

Next, a second modification of the first embodiment will be described. In the second modification, the index value calculation unit 618 first calculates the average value Rat_{AV} of the ratios in the predetermined region Rs. Then, the index value calculation unit 618 converts the average value Rat_{AV} using the function F1 to calculate the score SCp. The score SCp is the index value In. Then, the index value display control unit 619 displays the score SCp as the index value In relating to the elasticity (hardness or softness) of the body tissue on the display unit 7.

Next, a third modification of the first embodiment will be described. In the third modification, the comparison value calculation unit 614 performs computing expressed by the following equation (1) instead of the calculation of the ratio Rat as the computing for comparing the strain St for each pixel with the strain average value St_{AV}.

|St−St_{AV}|/St_{AV} (1)

Then, the value obtained by equation (1) is used in place of the ratio Rat, thereby generating the gradation data and calculating the index value In.

Next, a fourth modification of the first embodiment will be described. In the fourth modification, two regions Rs1 and Rs2 are set in the elastic image EG as shown in FIG. 6. Then, the index value calculation unit 618 calculates the respective index values In1 and In2 for the regions Rs1 and Rs2, using any one of the above methods.

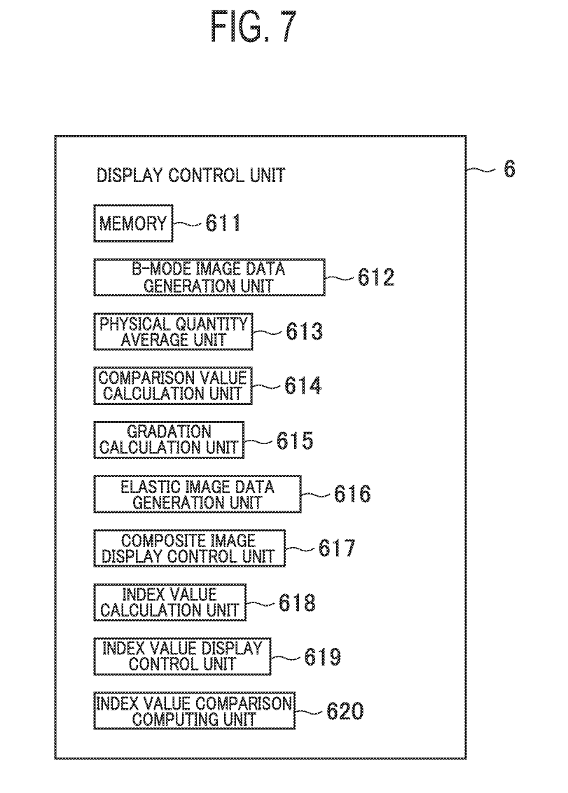

The display control unit 6 has an index value comparison computing unit 620 as shown in FIG. 7. The index value comparison computing unit 620 performs computing for comparing the index values In1 and In2. In this example, the ratio Rat_{I} between the index values In1 and In2 is calculated. The index value comparison computing unit 620 is one example of an embodiment of an index value comparison computing unit according to the invention. The index value display control unit 619 displays the ratio Rat_{I} and the index values In1 and In2 on the display unit 7.

According to this example, in the case where the regions Rs1 and Rs2 are set to, for example, a tumor portion and a fat portion, it is possible to know the quantified value of the hardness of the tumor with respect to the fat by referring to the ratio Rat_{I}.

Next, a fifth modification of the first embodiment will be described. In the fifth modification, the ratio Rat calculated for each pixel or the value calculated by equation (1) may be stored in the memory 611 or the HDD 10. In this case, by reading the ratio Rat or the value calculated by equation (1) which is stored in the memory 611 or the HDD 10, it is possible to redisplay the index value In calculated by a different method from the index value In displayed at the time of displaying the real-time ultrasonic image G.

### Second Embodiment

Next, a second embodiment will be described. This embodiment has the same basic configuration as the first embodiment, and therefore will be described employing the block diagram used in the first embodiment. Hereinafter, items different from those of the first embodiment will be described.

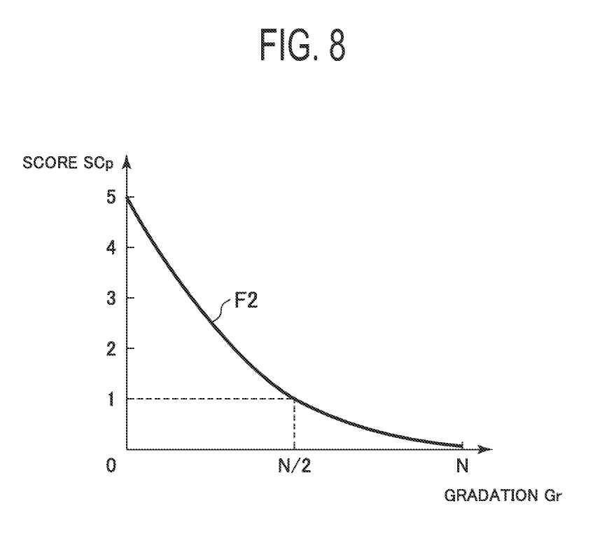

In this example, the index value calculation unit 618 converts the gradation Gr for each pixel into the score SCp using a function F2, thus calculating the score SCp for each pixel. In this example, the gradation Gr is converted into the score SCp in the range of 0.1 to 5 as in the first embodiment, using the function F2 as shown in FIG. 8. In FIG. 8, symbol N denotes the maximum gradation, and N=256 for example.

In this example too, lower scores SCp represent softer body tissues, and higher scores represent harder body tissues, as in the first embodiment.

In this example, the function F2 is a function in which the score SCp=1 at a gradation (N/2) which is one-half of the maximum gradation N. Further, the function F2 is a function in which the gradation of not lower than 0 and lower than N/2 is converted into the score SCp of higher than 1 and not higher than 5 and the gradation Gr of not lower than N/2 and not higher than N is converted into the score SCp of not lower than 0.1 and not higher than 1.

The range of gradations Gr lower than N/2 is the range of ratios Rat lower than 1, and is the range of strain values St lower than the average value St_{AV}. Therefore, in this example too, scores are distributed more minutely in the range of gradations Gr lower than N/2 which is the range of body tissue harder than average than in the range of gradations Gr not lower than N/2.

The index value calculation unit 618 calculates the average value SC_{AV} of the scores in the predetermined region Rs based on the score SCp for each pixel, as in the first embodiment. The average value SC_{AV} is the index value In. Then, the index value display control unit 619 displays the average value SC_{AV} as the index value In on the display unit 7.

According to the ultrasonic diagnostic apparatus 1 of this example, since the average value SC_{AV} is displayed as the index value In relating to the elasticity, it is possible to quantify and indicate the hardness or softness of the body tissue. Therefore, it is possible to make an objective and easy diagnosis.

Next, modifications of the second embodiment will be described. First, a first modification will be described. The index value calculation unit 618 calculates the average value Gr_{AV} of the gradations Gr of pixels in the predetermined region Rs, as the index value In. Then, the index value display control unit 619 displays the average value Gr_{AV} as the index value In relating to the elasticity (hardness or softness) of the body tissue on the display unit 7.

Next, a second modification of the second embodiment will be described. In the second modification, the index value calculation unit 618 first calculates the average value Gr_{AV} of the gradations Gr in the predetermined region Rs. Then, the index value calculation unit 618 converts the average value Gr_{AV} using the function F2 to calculate the score SCp. The score SCp is the index value In. Then, the index value display control unit 619 displays the score SCp as the index value In relating to the elasticity (hardness or softness) of the body tissue on the display unit 7.

Next, a third modification of the second embodiment will be described. In the third modification of the second embodiment, as in the fourth modification of the first embodiment, the two regions Rs1 and Rs2 are set in the elastic image EG (see FIG. 6). Then, the index value calculation unit 618 calculates the respective index values In1 and In2, based on gradations Gr, for the regions Rs1 and Rs2, using any one of the above methods.

Further, as with the display control unit 6 according to the fourth modification of the first embodiment, the display control unit 6 according to the third modification of the second embodiment also has the index value comparison computing unit 620 (see FIG. 7). The index value comparison computing unit 620 calculates the ratio Rat_{I} between the index values In1 and In2 calculated based on the gradations Gr. Then, the index value display control unit 619 displays the ratio Rat_{I} and the index values In1 and In2 on the display unit 7.

### Third Embodiment

Next, a third embodiment will be described. Hereinafter, configurations different from those of the first and second embodiments will be described.

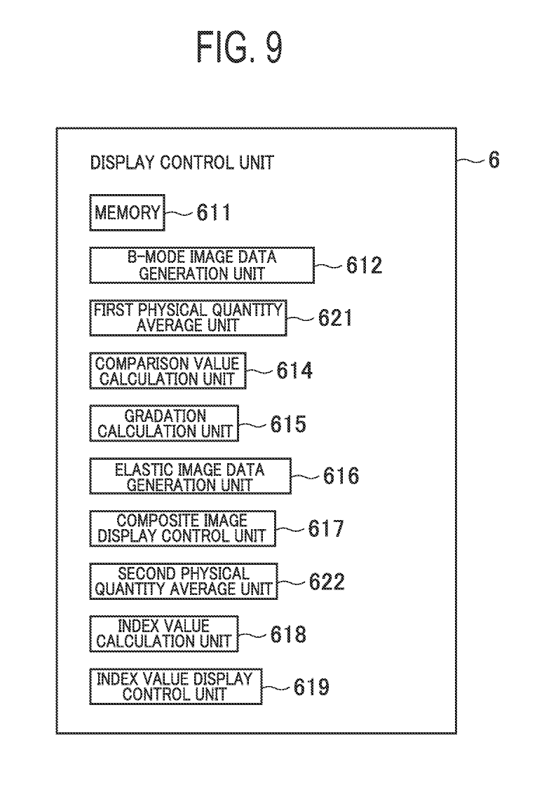

In this example, the display control unit 6 has the memory 611, the B-mode image data generation unit 612, a first physical quantity average unit 621, the comparison value calculation unit 614, the gradation calculation unit 615, the elastic image data generation unit 616, the composite image display control unit 617, a second physical quantity average unit 622, the index value calculation unit 618, and the index value display control unit 619, as shown in FIG. 9.

The first physical quantity average unit 621 calculates a first average value St_{AV}1 which is the average value of the strains in the elastic image display region Re (see FIGS. 3 and 4) based on the physical quantity data composed of data of the strain for each pixel, as with the physical quantity average unit 613 according to the first and second embodiments. In this example, the comparison value calculation unit 614 calculates the ratio Rat, using the first average value St_{AV}1 in place of the comparison value St_{AV}.

Further, the second physical quantity average unit 622 calculates a second average value St_{AV}2 which is the average value of strains in the predetermined region Rs (see FIG. 4) set in the elastic image EG. The first physical quantity average unit 621 is one example of an embodiment of a first physical quantity average unit, and the second physical quantity average unit 622 is one example of an embodiment of a second physical quantity average unit. Further, the first average value St_{AV}1 is one example of an embodiment of a first physical quantity average value, and the second average value St_{AV}2 is one example of an embodiment of a second physical quantity average value.

In this example, the index value calculation unit 618 performs computing for comparing the first average value St_{AV}1 and the second average value St_{AV}2 to calculate a comparison value as an index value In for the predetermined region Rs. Specifically, the index value calculation unit 618 calculates the ratio (SRat1=St_{AV}2/St_{AV}1) of the second average value St_{AV}2 to the first average value St_{AV}1, as the comparison value. Then, the index value display control unit 619 displays the ratio SRat1 as the index value In on the display unit 7. In the case where the predetermined region Rs is set to a tumor portion, the second average value St_{AV}2 is the average value of the strains of the tumor, so that it is possible to know the quantified value of the tumor by the index value In.

According to the ultrasonic diagnostic apparatus 1 of this example, since the ratio SRat1 is displayed as the index value In relating to the elasticity, it is possible to quantify and indicate the hardness or softness of the body tissue. Therefore, it is possible to make an objective and easy diagnosis.

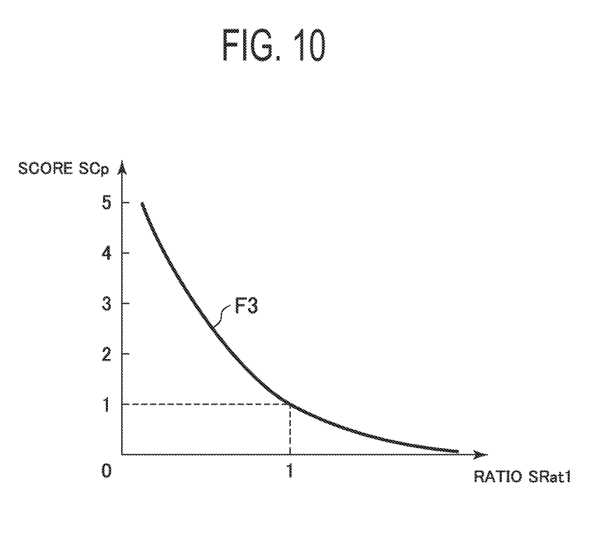

Next, modifications of the third embodiment will be described. First, a first modification will be described. In this example, the index value calculation unit 618 converts the ratio SRat1 using a predetermined function to calculate the score SCp as the index value In. In this example, the ratio SRat1 is converted into the score SCp in the range of 0.1 to 5 as in the first and second embodiments, using a function F3 as shown in FIG. 10. The function F3 is a function in which if the ratio SRat1 is 1, that is, the second average value St_{AV}2 is equal to the first average value St_{AV}1, the score SCp is 1, as in the first embodiment. Further, the function F3 is a function in which the ratio SRat1 of lower than 1 is converted into the score SCp of higher than 1 and not higher than 5 and the ratio SRat1 of not lower than 1 is converted into the score SCp of not lower than 0.1 and not higher than 1, as in the first embodiment.

The index value display control unit 619 displays the score SCp as the index value In on the display unit 7.

Next, a second modification of the third embodiment will be described. In the second modification, the index value calculation unit 618 performs computing expressed by the following equation (2) instead of the calculation of the ratio SRat1 as the computing for comparing the first average value St_{AV}1 and the second average value St_{AV}2.

|St_{AV}2−St_{AV}1|/St_{AV}1 (2)

Then, the index value display control unit 619 displays the value obtained by equation (2) as the index value In in place of the ratio SRat1. Further, based on the value obtained by equation (2), the score SCp may be calculated.

Next, a third modification of the third embodiment will be described. In the third modification of the third embodiment, as in the fourth modification of the first embodiment and the third modification of the second embodiment, the two regions Rs1 and Rs2 are set in the elastic image (see FIG. 6). Then, the index value calculation unit 618 calculates the respective index values In1 and In2 for the regions Rs1 and Rs2, using any one of the above methods.

Further, as with the display control unit 6 according to the fourth modification of the first embodiment and the third modification of the second embodiment, the display control unit 6 according to the third modification of the third embodiment also has the index value comparison computing unit 620 (see FIG. 7). The index value comparison computing unit 620 calculates the ratio Rat_{I} between the index values In1 and In2. Then, the index value display control unit 619 displays the ratio Rat_{I} and the index values In1 and In2 on the display unit 7.

### Fourth Embodiment

Next, a fourth embodiment will be described. Hereinafter, configurations different from those of the first to third embodiments will be described.

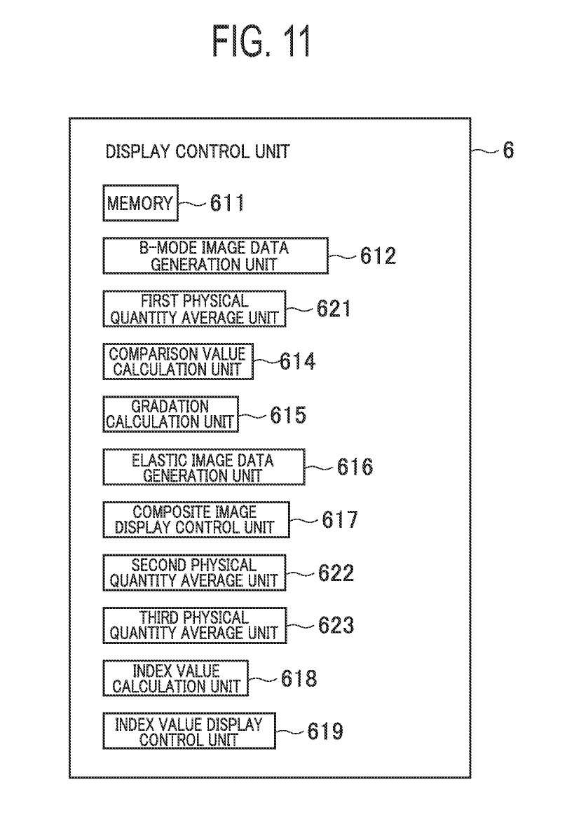

In this example, the display control unit 6 has the memory 611, the B-mode image data generation unit 612, the first physical quantity average unit 621, the comparison value calculation unit 614, the gradation calculation unit 615, the elastic image data generation unit 616, the composite image display control unit 617, the second physical quantity average unit 622, a third physical quantity average unit 623, the index value calculation unit 618, and the index value display control unit 619, as shown in FIG. 11.

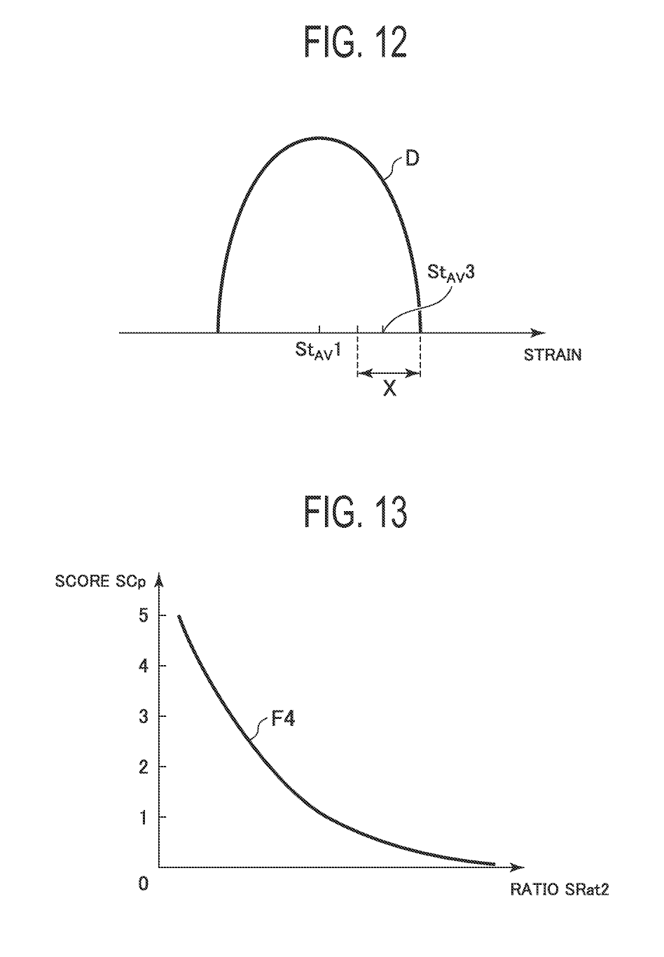

The third physical quantity average unit 623 calculates a third average value St_{AV}3 which is the average value of strains in a predetermined range in the distribution of strains calculated for pixels in the elastic image display region Re. The predetermined range is set on a higher strain side or a lower strain side than the first average value St_{AV}1 (softer side or harder side of the body tissue than average). A specific description will be made with reference to FIG. 12. In FIG. 12, a distribution D represents the distribution of strains calculated for pixels in the elastic image display region Re. The third physical quantity average unit 623 calculates the third average value St_{AV}3 which is the average value of strains in a predetermined strain range X of strain values higher than the first average value St_{AV}1, in the distribution D. The third physical quantity average unit 623 is one example of an embodiment of a third physical quantity average unit, and the third average value St_{AV}3 is one example of an embodiment of a third physical quantity average value.

The predetermined strain range X is a range indicating that the body tissue is softer than the average in the elastic image display region Re. For example, in the elastic image of a mammary gland region, by setting the elastic image display region Re to an appropriate range including fat, the predetermined strain range X can be set to softness mainly dominated by the fat tissue. In this case, the third average value St_{AV}3 is nearly the average value of the strains of the fat.

In this example, the index value calculation unit 618 performs computing for comparing the second average value St_{AV}2 and the third average value St_{AV}3 to calculate a comparison value as an index value In relating to the elasticity of the predetermined region Rs. Specifically, the index value calculation unit 618 calculates the ratio (SRat2=St_{AV}2/St_{AV}3) of the second average value St_{AV}2 to the third average value St_{AV}3, as the comparison value. Then, the index value display control unit 619 displays the ratio SRat2 as the index value In on the display unit 7. In the case where the predetermined region Rs is set to a tumor portion and the third average value St_{AV}3 is the average value of the strains of the fat, it is possible to know the quantified value of the hardness of the tumor with respect to the fat by the index value In.

According to the ultrasonic diagnostic apparatus 1 of this example, since the ratio SRat2 is displayed as the index value In relating to the elasticity, it is possible to quantify and indicate the hardness or softness of the body tissue. Therefore, it is possible to make an objective and easy diagnosis.

Next, modifications of the fourth embodiment will be described. First, a first modification will be described. In this example, the index value calculation unit 618 converts the ratio SRat2 using a predetermined function to calculate the score SCp as the index value In. In this example, the ratio SRat2 is converted into the score SCp in the range of 0.1 to 5 as in the first to third embodiments, using a function F4 as shown in FIG. 13.

The index value display control unit 619 displays the score SCp as the index value In on the display unit 7.

Next, a second modification of the fourth embodiment will be described. In the second modification, the index value calculation unit 618 performs computing expressed by the following equation (3) as the computing for comparing the second average value St_{AV}2 and the third average value St_{AV}3, instead of the calculation of the ratio SRat2.

(St_{AV}3−St_{AV}2)/St_{AV}3 (3)

Then, the index value display control unit 619 displays the value obtained by equation (3) as the index value In in place of the ratio SRat2. Further, based on the value obtained by equation (3), the score SCp may be calculated.

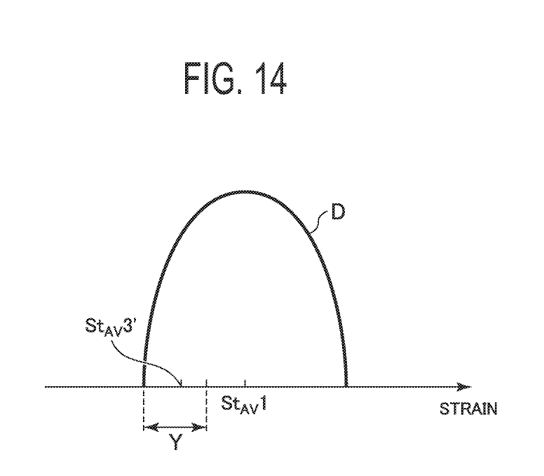

Next, a third modification of the fourth embodiment will be described. In the case where the predetermined region Rs is set to a fat portion, the third physical quantity average unit 623 calculates a third average value St_{AV}3′ which is the average value of strains in a predetermined strain range Y of strain values lower than the first average value St_{AV}1, in the distribution D shown in FIG. 14. The predetermined strain range Y is a range indicating that the body tissue is harder than the average in the elastic image display region Re, and can be set to the hardness of a tumor by setting the elastic image display region Re to an appropriate range including the tumor in the elastic image.

In the third modification, the predetermined region Rs is set to a portion to be compared with the third average value St_{AV}3′ such as the fat portion.

The index value calculation unit 618 calculates the ratio (SRat2′=St_{AV}3′/St_{AV}2) of the third average value to the second average value, as a comparison value. Then, the index value display control unit 619 displays the ratio SRat2′ as an index value In on the display unit 7.

The index value calculation unit 618 may perform computing expressed by the following equation (3′) instead of the ratio SRat2′.

(St_{AV}2−St_{AV}3′)/St_{AV}2 (3′)

Further, the index value calculation unit 618 may calculate the score SCp, based on the ratio SRat2′ and the value calculated by equation (3′).

Next, a fourth modification of the fourth embodiment will be described. In the fourth modification of the fourth embodiment, as in the fourth modification of the first embodiment and the third modifications of the second and third embodiments, the two regions Rs1 and Rs2 are set in the elastic image (see FIG. 6). Then, the index value calculation unit 618 calculates the respective index values In1 and In2 for the regions Rs1 and Rs2, using any one of the above methods.

Further, as with the display control unit 6 according to the fourth modification of the first embodiment and the third modifications of the second and third embodiments, the display control unit 6 according to the fourth modification of the fourth embodiment also has the index value comparison computing unit 620 (see FIG. 7). The index value comparison computing unit 620 calculates the ratio Rat_{I} between the index values In1 and In2. Then, the index value display control unit 619 displays the ratio Rat_{I} and the index values In1 and In2 on the display unit 7.

### Fifth Embodiment

Next, a fourth embodiment will be described. Hereinafter, configurations different from those of the first to fourth embodiments will be described.

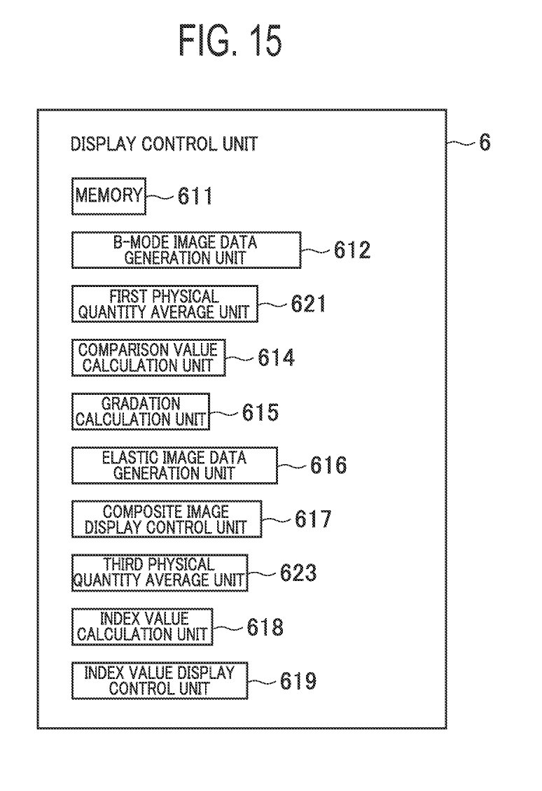

In this example, the display control unit 6 has the memory 611, the B-mode image data generation unit 612, the first physical quantity average unit 621, the comparison value calculation unit 614, the gradation calculation unit 615, the elastic image data generation unit 616, the composite image display control unit 617, the third physical quantity average unit 623, the index value calculation unit 618, and the index value display control unit 619, as shown in FIG. 15.

In this example, the third physical quantity average unit 623 calculates the third average value St_{AV}3′ which is the average value of the strains in the predetermined strain range Y, in the distribution D shown in FIG. 14.

Further, the index value calculation unit 618 performs computing for comparing the first average value St_{AV}1 and the third average value St_{AV}3′ to calculate a comparison value as an index value In relating to the elasticity. Specifically, the index value calculation unit 618 calculates the ratio (SRat3=St_{AV}3′/St_{AV}1) of the third average value St_{AV}3′ to the first average value St_{AV}1, as the comparison value. Then, the index value display control unit 619 displays the ratio SRat3 as the index value In on the display unit 7. In the case where the third average value St_{AV}3′ is the average value of the strains of the tumor, it is possible to know the quantified value of the hardness of the tumor by the index value In.

According to the ultrasonic diagnostic apparatus 1 of this example, since the ratio SRat3 is displayed as the index value In relating to the elasticity, it is possible to quantify and indicate the hardness or softness of the body tissue. Therefore, it is possible to make an objective and easy diagnosis.

Next, modifications of the fifth embodiment will be described. First, a first modification will be described. In this example, the index value calculation unit 618 converts the ratio SRat3 using a predetermined function to calculate the score SCp as the index value In. In this example, a function (not shown) in which the ratio SRat3 is converted into the score SCp of higher than 1 and not higher than 5 is used as the predetermined function.

The index value display control unit 619 displays the score SCp as the index value In on the display unit 7.

Next, a second modification of the fifth embodiment will be described. In the second modification, the index value calculation unit 618 performs computing expressed by the following equation (4) instead of the calculation of the ratio SRat3 as the computing for comparing the first average value St_{AV}1 and the third average value St_{AV}3′.

(St_{AV}1−St_{AV}3′)/St_{AV}3′ (4)

Then, the index value display control unit 619 displays the value obtained by equation (4) as the index value In in place of the ratio SRat3. Further, based on the value obtained by equation (4), the score SCp may be calculated.

Next, a third modification of the fifth embodiment will be described. The average value to be compared with the first average value St_{AV}1 is not limited to the third average value St_{AV}3′, and may be an average value in an arbitrarily-set predetermined strain range.

### Sixth Embodiment

Next, a sixth embodiment will be described. Hereinafter, configurations different from those of the first to fifth embodiments will be described.

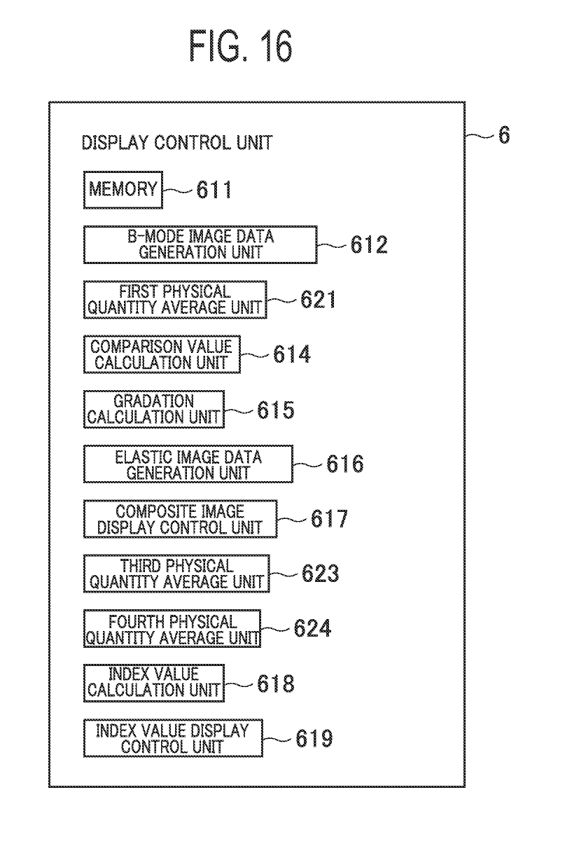

In this example, the display control unit 6 has the memory 611, the B-mode image data generation unit 612, the first physical quantity average unit 621, the comparison value calculation unit 614, the gradation calculation unit 615, the elastic image data generation unit 616, the composite image display control unit 617, the third physical quantity average unit 623, a fourth physical quantity average unit 624, the index value calculation unit 618, and the index value display control unit 619, as shown in FIG. 16.

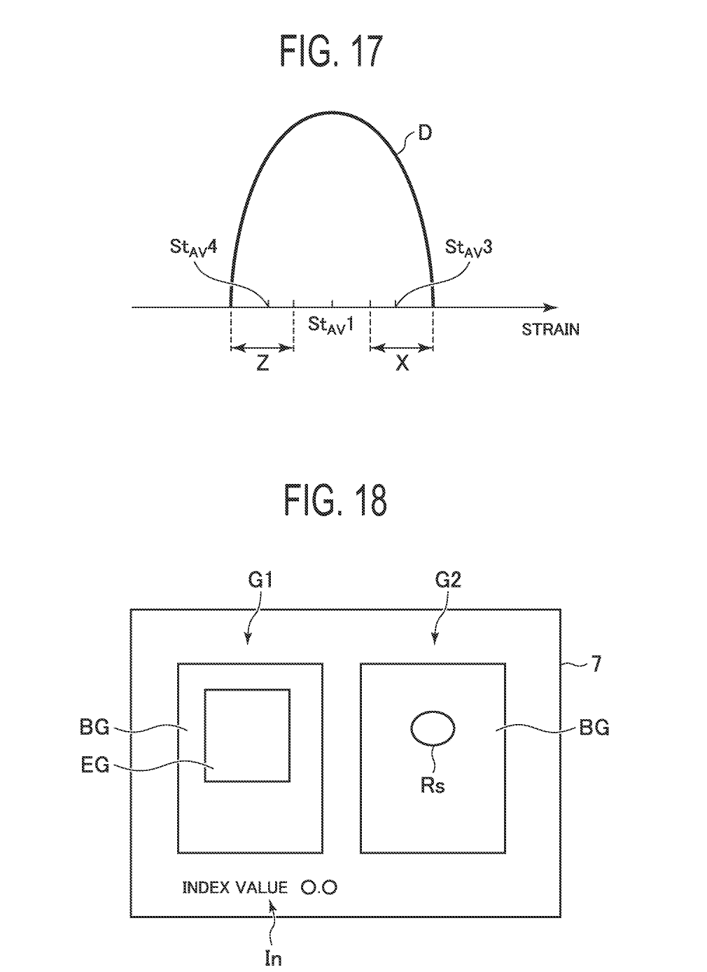

In this example, the third physical quantity average unit 623 calculates the third average value St_{AV}3 which is the average value of the strains in the predetermined strain range X, in the distribution D shown in FIG. 17. The predetermined strain range X is the same as that of FIG. 12.

Further, the fourth physical quantity average unit 624 calculates a fourth average value St_{AV}4 which is the average value of the strains in the predetermined strain range Z, in the distribution D shown in FIG. 17. The predetermined strain range Z is the same as the predetermined strain range Y shown in FIG. 14, and the fourth average value St_{AV}4 is the same as the third average value St_{AV}3′. The fourth physical quantity average unit 624 is one example of an embodiment of a fourth physical quantity average unit, and the fourth average value St_{AV}4 is one example of an embodiment of a fourth physical quantity average value.

The index value calculation unit 618 performs computing for comparing the third average value St_{AV}3 and the fourth average value St_{AV}4 to calculate a comparison value as an index value In relating to the elasticity. Specifically, the index value calculation unit 618 calculates the ratio (SRat4=St_{AV}4/St_{AV}3) of the fourth average value St_{AV}4 to the third average value St_{AV}3, as the comparison value. Then, the index value display control unit 619 displays the ratio SRat4 as the index value In on the display unit 7. In the case where the third average value St_{AV}3 is the average value of the strains of the fat and the fourth average value St_{AV}4 is the average value of the strains of the tumor, it is possible to know the quantified value of the hardness of the tumor with respect to the fat by the index value In.

According to the ultrasonic diagnostic apparatus 1 of this example, since the ratio SRat4 is displayed as the index value In relating to the elasticity, it is possible to quantify and indicate the hardness or softness of the body tissue. Therefore, it is possible to make an objective and easy diagnosis.

Next, modifications of the sixth embodiment will be described. First, a first modification will be described. The index value calculation unit 618 converts the ratio SRat4 using a predetermined function to calculate the score SCp as the index value In. In this example, for example, a function (not shown) in which the ratio SRat4 is converted into the score SCp in the range of 0.1 to 5 as in the first to fourth embodiments is used.

The index value display control unit 619 displays the score SCp as the index value In on the display unit 7.

Next, a second modification of the sixth embodiment will be described. In the second modification, the index value calculation unit 618 performs computing expressed by the following equation (5) instead of the calculation of the ratio SRat4 as the computing for comparing the third average value St_{AV}3 and the fourth average value St_{AV}4.

(St_{AV}3−St_{AV}4)/St_{AV}3 (5)

Then, the index value display control unit 619 displays the value obtained by equation (5) as the index value In in place of the ratio SRat4. Further, based on the value obtained by equation (5), the score SCp may be calculated.

While the invention has been described with reference to the above embodiments, the invention can, of course, be changed in various ways without departing from the gist thereof. For example, as shown in FIG. 18, an image G1 composed of the B-mode image BG and the elastic image EG and an image G2 composed of only the B-mode image BG may be displayed side-by-side on the display unit 7. In this case, the images G1 and G2 are images in the same portion of the body tissue. Further, the predetermined region Rs is set in the image G2, and the index value In for the predetermined region Rs is displayed.

Further, in the calculation of the ratios Rat, SRat1, SRat2, SRat2′, SRat3, and SRat4, the numerator and the denominator may be reversed.

Furthermore, in the above embodiments, instead of the strain, a displacement due to the deformation of the body tissue, an elastic modulus, or the like may be calculated as the physical quantity relating to the elasticity of the body tissue.

1. An ultrasonic diagnostic apparatus comprising:
a physical quantity calculation unit configured to calculate a physical quantity relating to an elasticity of body tissue based on echo data acquired by transmitting ultrasound to and receiving ultrasound from the body tissue;
a physical quantity average unit configured to calculate an average value of physical quantities in an elastic image of the body tissue generated based on the calculated physical quantity;
a comparison value calculation unit configured to calculate a comparison value for each pixel by comparing a physical quantity for each pixel of the elastic image with the average value; and
an index value calculation unit configured to calculate an index value relating to an elasticity of a predetermined region in the elastic image based on the comparison values.

2. The ultrasonic diagnostic apparatus according to claim 1, wherein the index value calculation unit is configured to calculate a score for each pixel by converting the comparison value using a predetermined function, and to calculate an average value of scores in the predetermined region as the index value.

3. The ultrasonic diagnostic apparatus according to claim 1, wherein the index value calculation unit is configured to calculate an average value of comparison values in the predetermined region as the index value.

4. The ultrasonic diagnostic apparatus according to claim 1, wherein the index value calculation unit is configured to calculate an average value of comparison values in the predetermined region, and to calculate a score as the index value by converting the average value using a predetermined function.

5. An ultrasonic diagnostic apparatus comprising:
a physical quantity calculation unit configured to calculate a physical quantity relating to an elasticity of body tissue based on echo data acquired by transmitting ultrasound to and receiving ultrasound from the body tissue;
a gradation calculation unit configured to calculate a gradation for each pixel by performing gradation processing based on the physical quantity for each pixel of an elastic image of the body tissue generated based on the physical quantities; and
an index value calculation unit configured to calculate an index value relating to an elasticity of a predetermined region based on the gradations.

6. The ultrasonic diagnostic apparatus according to claim 5, wherein the index value calculation unit is configured to calculate a score for each pixel by converting the gradation using a predetermined function, and to calculate an average value of scores in the predetermined region as the index value.

7. The ultrasonic diagnostic apparatus according to claim 5, wherein the index value calculation unit is configured to calculate an average value of gradations in the predetermined region as the index value.

8. The ultrasonic diagnostic apparatus according to claim 5, wherein the index value calculation unit is configured to calculate an average value of gradations in the predetermined region, and to calculate a score as the index value by converting the average value using a predetermined function.

9. An ultrasonic diagnostic apparatus comprising:
a physical quantity calculation unit configured to calculate a physical quantity relating to an elasticity of body tissue based on echo data acquired by transmitting ultrasound to and receiving ultrasound from the body tissue;
a first physical quantity average unit configured to calculate a first physical quantity average value which is an average value of physical quantities in an elastic image of the body tissue generated based on the physical quantity;
a second physical quantity average unit configured to calculate a second physical quantity average value comprising an average value of physical quantities in a predetermined region within the elastic image; and
an index value calculation unit configured to calculate a comparison value as an index value relating to an elasticity of the predetermined region by comparing the first physical quantity average value and the second physical quantity average value.

10. The ultrasonic diagnostic apparatus according to claim 9, wherein the index value calculation unit is configured to calculate a score as the index value by converting the comparison value using a predetermined function.

11. The ultrasonic diagnostic apparatus according to claim 1, further comprising an index value comparison computing unit configured to compare index values for a plurality of predetermined regions.

12. The ultrasonic diagnostic apparatus according to claim 2, further comprising an index value comparison computing unit configured to compare index values for a plurality of predetermined regions.

13. The ultrasonic diagnostic apparatus according to claim 3, further comprising an index value comparison computing unit configured to compare index values for a plurality of predetermined regions.

14. The ultrasonic diagnostic apparatus according to claim 4, further comprising an index value comparison computing unit configured to compare index values for a plurality of predetermined regions.

15. The ultrasonic diagnostic apparatus according to claim 5, further comprising an index value comparison computing unit configured to compare index values for a plurality of predetermined regions.

16. The ultrasonic diagnostic apparatus according to claim 6, further comprising an index value comparison computing unit configured to compare index values for a plurality of predetermined regions.

17. The ultrasonic diagnostic apparatus according to claim 7, further comprising an index value comparison computing unit configured to compare index values for a plurality of predetermined regions.

18. The ultrasonic diagnostic apparatus according to claim 9, comprising an index value comparison computing unit for performing computing for comparing index values for a plurality of predetermined regions.

19. The ultrasonic diagnostic apparatus according to claim 1, comprising an index value display control unit configured to display the index value.

20. The ultrasonic diagnostic apparatus according to claim 5, comprising an index value display control unit configured to display the index value.
